(19) United States
(12) Patent Application Publication
(10) Pub. No.: US 2014/0037606 A1
(43) Pub. Date: Feb. 6, 2014

(54) CELL-BASED, ANTI-CANCER VACCINES

(71) Applicant: Eyal Amiel, Hinesburg, VT (US)

(72) Inventor: Eyal Amiel, Hinesburg, VT (US)

(21) Appl. No.: 13/960,734

(22) Filed: Aug. 6, 2013

Related U.S. Application Data
(60) Provisional application No. 61/680,224, filed on Aug. 6, 2012.

(51) Int. Cl. C12N 5/078

(52) U.S. Cl. CPC C12N 5/0634
USPC 424/93.71; 435/375; 435/372

(57) ABSTRACT

The present invention relates to cancer vaccines and more particularly to compositions and methods for producing activated antigen presenting cells (dendritic cells, macrophages, monocytes, or other cells capable of presenting antigen to T lymphocytes); to pharmaceutical compositions including such cells; and to methods of using such cells (e.g., in treating patients who are suffering from or at risk of developing cancer).

### CROSS-REFERENCE TO RELATED APPLICATIONS

This application claims the benefit of the filing date of U.S. Application No. 61/680,224, which was filed on Aug. 6, 2012, and which is incorporated herein by reference in its entirety.

### STATEMENT REGARDING FEDERALLY FUNDED RESEARCH

This invention was made with government support under grant numbers AI053825 and AI049823 awarded by the National Institutes of Health. The government has certain rights in the invention.

### FIELD OF THE INVENTION

The present invention relates to cancer vaccines and more particularly to compositions and methods for producing activated antigen presenting cells (dendritic cells, macrophages, monocytes, or other cells capable of presenting antigen to T lymphocytes); to pharmaceutical compositions including such cells; and to methods of using such cells (e.g., in treating patients who are suffering from or at risk of developing cancer).

### SUMMARY

In a first aspect, the present invention features methods of making an activated antigen presenting cell (e.g., a dendritic cell, macrophage, monocyte, or any other cell that presents or is capable of presenting an antigen to a T lymphocyte (m, for example, the context of eliciting antibody production)). The method can be carried out by exposing the antigen presenting cell (APC) to: an inhibitor of the mTOR signaling axis; an agonist that elicits APC activation (e.g. a Toll-like receptor agonist, a CD40 agonist, and the like); and a cancer-related antigen. The exposure can occur ex vivo (e.g., in a cell culture), and the APC can be obtained from a variety of sources. For example, an APC may be a mammalian APC (e.g., a dendritic cell, macrophage, or monocyte from a human, a non-human primate, or a bovine, canine, porcine, feline, murine, or equine animal). In any embodiment, the APC can be isolated from an environment in which it naturally resides (e.g., isolated from the blood or a tissue of a mammal). As an example, for generating human DCs, cells can be isolated directly from human blood samples. For clinical, research, and other applications, leukophoresis on patient blood can be performed to obtain an enriched population of circulating PBMCs, which can be further purified as indicated below. It is more difficult to extract APCs from tissue sources, but the use of such cells is within the scope of the present invention. Thus, the compositions and methods of the invention can be practiced with APCs isolated from tissue or from a blood sample or other body fluid. Other useful sources of APCs are cell subsets that are differentiated in vitro from circulating blood monocytes. Methods for obtaining, providing, or isolating APCs from a source, whether a living body or a cell culture environment, are known in the art.

In the studies described below, human myeloid DCs were isolated from human blood using MACS positive selection beads, and these methods can be employed in the context of the present invention. For clinical applications, negative selection or cell sorting may be preferable as a purification method, but the invention is not so limited. Blood cells can be obtained by reverse flushing filters in sterile Hank's Balanced Salt Solution (HBSS; or another physiologically acceptable salt solution). PBMCs can be obtained by centrifugation of blood samples over Ficoll-Paque Plus (density 1.077 g/mL) (GE Healthcare). Myeloid DCs can be enriched using the BDCA-1 Positive Selection DC Islation Kit (Miltenyi Bioscience) per the manufacturer's instructions. Freshly isolated DC (1×10⁵ cells/well in 200 μl) can be cultured in a suitable medium such as complete RPMI 1640 medium containing 10% FCS, 100 U/mL penicillin/streptomycin, and supplemented with 20 ng/mL GM-CSF (granulocyte macrophage colony-stimulating factor). This medium is suitable for use with other types of APCs. Similarly, DCs and other types of APCs can be stimulated simultaneously with 1 μg/mL R848 (a Toll-like receptor (TLR) agonist) in the presence or absence of mTOR inhibitors (both used at 100 nM).

The purity of the cell population subjected to the present methods, which we may refer to as a “target cell population”, can vary. For clinical applications, at least 75% purity is likely to be preferable. For example, within a given composition (e.g., a mixed population of cells) the desired cell type (i.e., an APC) can constitute at least or about 80%, 85%, 90%, 95%, 98% 99% or a greater percentage of the composition. There is evidence that mTOR inhibition has a positive effect on the activation of myeloid DCs but a negative effect on the activation of classically defined monocyte-derived human DCs (i.e., monocytes cultured for 7 days in GM-CSF+IL-4 culture conditions). However, different subsets of DCs cultured from monocytes can be established by the addition of other cytokines during differentiation (e.g. TNF-alpha, IFN-gamma, IL-6, PGE-2, and others). Our data suggest that resident myeloid DCs (BDCA-1⁺ subset naturally present and circulating in human blood) may be a particularly attractive candidate for this approach (see FIG. 3), and can therefore be incorporated in any of the compositions and methods described herein. The present invention encompasses methods in which any variety of DC that can be differentiated from human blood cells can be exposed to the combination of one or more mTOR inhibitors; one or more agonists of a Toll-like receptor; and one or more cancer antigens. As noted, the DC can be a subset cultured from monocytes or “freshly” isolated.

The inhibitor of mTOR can be rapamycin or an analog thereof (e.g., sirolimus, everolimus, ridaforolimus, temsirolimus, umirolimus, or zotarolimus). Other useful analogs, which may be employed alone or in combination, are in the class of ATP-competitive inhibitors of mTOR function (e.g., Torin1 and Torin2). In some embodiments, EGCG, caffeine, curcumin, and resveratrol may be used as well, although their use could be hampered by non-specific effects. Furthermore, inhibitors of signaling molecules upstream of mTOR its (e.g. inhibitors of phosphatidylinositide 3-kinases and Protein Kinase B) show similar results to mTOR inhibitors in our experimental system regarding their effect on APC activation.

In any of the methods of making an activated APC (or other methods described herein which employ an agonist such as a TLR), the TLR agonist can be an antibody (or a variant or fragment thereof) that specifically binds activating receptors on the APC. The antibody moiety, which we may refer to more simply as an “antibody”, can be a naturally occurring antibody (such as a tetrameric immunoglobulin of the G class) or a biologically active fragment or variant thereof. The antibody moiety can also be a single chain antibody (scFv), di-scFv, single domain antibody (sdAb), affibodies, affilin, anticalin, avimer, DARPin, fynomer, Kunitz domain peptide, or monobody. Where the antibody moiety is a biologically active fragment of a larger antibody, it can be an Fab, Fab′2 or F(ab′)2 fragment.

Activation of a number of receptors can activate APCs, and these receptors can be targeted (e.g., with an antibody) as described herein. There are at least ten human TLRs, each demonstrating specificity for a specific set of ligands. TLR3, TLR4, TLR5, TLR7, TLR8 and TLR9 naturally recognize double-stranded RNA (dsRNA), LPS of Gram-negative bacteria, bacterial flagellin, imidazoquinolines, single-stranded RNA, and bacterial or viral CpG DNA motifs, respectively. Moreover, TLR2 can heterodimerize with TLR1 or TLR6, to recognize peptidoglycan, lipopeptide, and lipoproteins. Likewise, TLR10 is can heterodimerize with TLR1 or TLR2 but the ligands of these heterodimers are unknown. Therefore activation of APCs can be brought about using any of the above ligands or antibodies to any of the receptors described above. In any of the methods of making an activated APC (or other methods described herein which employ an agonist such as a TLR), the agonist of the TLR can be a TLR ligand (e.g., lipopolysaccharide (LPS), optionally from a bacterium such as E. coli.). Because we have found a beneficial effect of mTOR inhibition with all TLR ligands tested in the mouse system, we expect the methods described herein to achieve APC (e.g., DC) activation when employing any one or more TLR ligands, including any one or more currently known in the art. These include TLR2, TLR3, TLR4, TLR5, TLR7/8, and TLR9 ligands for activation of DCs. Alternatively, non-TLR activation methods of inducing DC maturation, such as CD40 ligation, may be employed. In this approach, agonistic anti-CD40 antibodies or soluble CD40-ligand are used to activate CD40, an important costimulatory molecule on the surface of APCs that is required for their full activation.

We may often refer to an activated antigen-presenting cell, such as a dendritic cell. Unless the context clearly indicates otherwise, all references to a cell, in the singular, are equally applicable to populations of cells.

In any of the methods of making an activated APC (or other methods described herein which employ a cancer-related antigen), the cancer-related antigen can be an indicator of an epithelial cancer (e.g., melanoma). About 85% of all cancers are cancers involving epithelial cells, also known as carcinomas. More specifically, the cancer can be a squamous cell carcinoma, adenocarcinoma (of glandular cells), or transitional cell carcinoma, although some cancers are “mixed” (e.g., adenosquamous carcinoma). The cancers can be classified by tissue origin, and the most common form of carcinoma of the prostate is adenocarcinoma. More than 98% of all lung cancers are carcinomas; nearly all breast cancers are ductal carcinomas; nearly all malignancies of the colon and rectum are either adenocarcinoma or squamous cell carcinoma; pancreatic carcinoma is almost always of the adenocarcinoma type and is highly lethal. The cancer-related antigen, which may be more simply referred to as a cancer antigen, can also be an indicator of a central nervous system cancer (including gliomas); of a reproductive organ cancer (e.g., a Mullerian-derived cancer or a prostate cancer); of a mesothelioma; of pancreatic cancer; of non-small cell lung carcinoma or other carcinomas of the respiratory tract; of squamous cell carcinoma; of an adenocarcinoma; of a renal cancer; of a gastrointestinal cancer (such as stomach cancer or colon cancer); a cancer of the mouth, tongue, or eye; a liver cancer; or of a hematologic malignancy (e.g., a leukemia, lymphoma, myeloma, or other hematologic malignancy).

In any of the present methods, the cancer antigen can be: A3, AFP, AKAP-4, ALK, androgen receptor, B7H3, Bcr-abl, BORIS, BR-1, BRAC1, BRAC2, carbonic anhydrase IX, CEA, cyclin B1, CYP1B1, EGFRviii, EpCAM, EphA2, ERG, ESO-1, ETV6-AML, FAP, fos-related antigen 1, fucosyl GMT, GD2, GD3, GMe ganglioside, GloboH, gp100, HER-2/neu, HMWMAA, HPV E6 or E7, hTERT, LCK, legumain, LMP2, MAC-CT-1, MAD-CT-2, MAGE, MAGE A1, MelanA/MART1, mesothelin, ML-IAP, MUC1, MYCN, NA17, NY-RGS5, NY-Ras-mutant, OY-TES™, p53, Page4, PAP, PAX3, PAX5, PDGFR-beta, PLAC1, polysialic acid, proteinase3, PSA, PSCA, PSMA, RhoC, SART3, sLe(a), sperm protein 17, a sperm fibrous sheath protein, SSX2, STn, survivin, Tie 2, Tn, TRP-1, TRP-2, tyrosinase, VGFR2, WT1, or XAGE1. The cancer antigen can be isolated from the patient to be treated. However, because there may be a danger of loading APCs with too much off-target “self” antigen, the present methods can also employ cancer antigens provided from an exogenous source. For example, the cancer antigen can be a recombinant protein or peptide. In some embodiments, the APCs can be exposed to (e.g., co-incubated with) tumor lysates. In some embodiments, the APCs can be exposed to (e.g., co-incubated with) a recombinant cancer antigen.

Where a cancer antigen is exclusively expressed by a tumor cell, that antigen can be used in the methods described herein to produce a vaccine for prophylactic use, and the invention further encompasses methods of individualized or personalized treatments in which a cancer antigen is provided from a cancer patient, who is then treated as described herein (e.g., by administering to the patient an APC that was activated ex vivo by exposure to an inhibitor of the mTOR (mammalian target of rapamycin) signaling axis; an agonist that mediates activation of the APC; and a cancer-related antigen (optionally one obtained from the patient)).

In one embodiment, the APC is exposed to the inhibitor of mTOR and/or to the agonist of a TLR prior to the time the dendritic cell is exposed to the cancer-related antigen. In another embodiment, the APC is exposed to the inhibitor of mTOR, the agonist of APC activation, and a cancer-related antigen essentially simultaneously. The experimental evidence collected to date indicates that mTOR inhibitors exert their beneficial effect on APC activation whether cells are pretreated with mTOR inhibitors (i.e., prior to TLR activation) or whether there is a simultaneous treatment with mTOR inhibitors and TLR stimulation. The benefits of mTOR inhibition on APC activation may be reduced when APC activation occurs 18-24 hours before mTOR inhibitors are added to the system

In another aspect, the invention features an activated APC made by a method described herein.

In another aspect, the invention features a pharmaceutically acceptable composition that includes an activated APC made by a method described herein. Such pharmaceutical compositions may be sterile; may contain only the activated APCs as an active ingredient; may contain both activated APCs and other pharmaceutically active agents; or may contain an excipient, adjuvant, or carrier.

In another aspect, the invention features a kit that includes an activated APC as described herein and instructions for use. The kit may also include compositions and reagents for culturing the APCs; a pharmaceutical or physiologically acceptable composition containing the APCs; and/or paraphernalia for administration of the APCs or compositions containing them.

In another aspect, the invention features methods of treating a patient who has cancer or a pre-cancerous condition (e.g., cellular dysplasia) with the activated APCs described herein. The cell may be made by any of the methods described herein. One of ordinary skill in the art will appreciate that the cancer antigen used in the cellular activation method must be a cancer antigen relevant to the type of cancer with which the patient is suffering. For example, antigen-presenting (e.g., dendritic) cells useful in the treatment of prostate cancer can be activated with the cancer antigen PSA; antigen-presenting (e.g., dendritic) cells useful in the treatment of ovarian cancer can be activated with the cancer antigen HE4 or CA-125; and so forth.

### BRIEF DESCRIPTION OF THE DRAWINGS

FIG. 1. Inhibition of mTOR expression prolongs DC lifespan and promotes expression of costimulatory molecules CD40 and CD86. (A) DCs were pulsed with Pam2CSK4, LPS, R848, or CpG for 6 hours and then washed and cultured in complete medium. Cell viability was monitored daily by FACS analysis of 7-AAD staining of CD11c⁺ cells. (B) Western blot for mTOR and β-actin protein in Luciferase hpRNA (Luc^{hp}) or mTOR hpRNA (mTOR^{hp})-transduced DCs. (C) DCs were stimulated with media alone, LPS, or rapamycin+LPS for 30 minutes. Cells were subsequently fixed and stained for phosphorylated S6 protein as a molecular readout for mTOR activation and analyzed by FACS. (D) Luc^{hp} and mTOR^{hp}-transduced DCs were left untreated or stimulated with LPS and monitored daily for cell viability by analysis of 7-AAD staining of CD11c⁺ cells. (E) Luc^{hp} or mTOR^{hp} DCs were cultured with or without LPS for 24 hours and analyzed by FACS for CD40 and CD86 expression. All graphs in this figure represent mean values of replicate wells; all experiments were performed at least twice with similar results.

FIG. 2. Pharmacological inhibition of mTOR augments DC lifespan and costimulatory molecule expression. (A) DCs were stimulated with LPS in the presence or absence of rapamycin (RAP) or KU (synthetic ATP-competitive inhibitor of mTOR). Cells were monitored daily for cell viability as described for FIG. 1. Data are presented as mean+/−SD of 4 independent experiments. (B) DCs were unstimulated or stimulated with LPS, RAP+LPS, or KU+LPS and analyzed by FACS 24 hours later for CD40 and CD86 expression. Data are representative of more than 4 independent experiments. (C) DCs were treated as in (B) and analyzed daily by FACS for CD40 and CD86 expression gated on live CD11c⁺ cells. Data are presented as mean+/−SD of 4 independent experiments. (D) DCs were treated as indicated and supernatants collected 24 hours later for analysis by Cytometric Bead Array for IL-12p70, TNFα, and IL-10. Data are presented as mean+/−SD of 2 independent experiments.

FIG. 3. Pharmacological inhibition of mTOR augments the duration of costimulatory molecule expression in human myeloid DCs. (A) Human myeloid DCs were either unstimulated or stimulated with R848 in the presence or absence of rapamycin (RAP) or KU. CD40 and CD86 expression was analyzed by FACS. Day 4 costimulatory molecule expression from a representative donor (5430) is depicted. (B) DCs were treated as in (A) and analyzed daily by FACS for CD40 and CD86 expression. (C) DCs were treated as in (A) and analyzed for viability at indicated times by FACS analysis of 7-AAD staining. For each treatment, data from 4-6 individual donors is depicted.

FIG. 4. mTOR inhibition affects activation-induced metabolic changes in mouse but not human DCs. (A) Mouse BMDCs were either left unstimulated, or treated with LPS in the presence or absence of rapamycin or KU as indicated and supernatants collected 48 hours later for analysis of glucose concentration (left) and lactate concentration (right). (B) Human myeloid DCs were either left unstimulated, or treated with R848 in the presence or absence of rapamycin or KU as indicated and supernatants collected 48 hours later for analysis of glucose concentration (left) and lactate concentration (right). Asterisks indicate statistically significant differences between groups (p<0.05). For all graphs data represent the mean+/−SD of data from at least 3 individual mice or donors.

FIG. 5. mTOR inhibition in DCs improves their ability to stimulate CD8 T cell responses. (A) DCs were treated as indicated for 24 hours after which cells were washed and replaced with normal media. 2, 3, or 4 days after activation, DCs were co-cultured at a 1:5 ratio with CFSE-labeled OT-I CD8⁺ T cells for 4 days. T-cell proliferation was determined by CFSE dilution within CD8⁺ cell population. Data are representative of 3 independent experiments. (B) DCs were treated as indicated for 24 hours and stained for CCR7 expression. Data is representative of two independent experiments. (C) 10 mice per group were immunized subcutaneously with DCs stimulated in vitro for 6 hours with LPS, LPS plus OVA, or rapamycin (RAP) plus LPS plus OVA. 7 days later, draining (popliteal) LNs were harvested and frequencies of Kb-OVA tetramer⁺ CD8⁺ cells were determined. FACS plots represents concatenated data from all 10 individual mice per group. Data are representative of more than 3 individual experiments. (D) Total numbers of tetramer⁺ cells from (C) were calculated. (E) Mice were immunized as in (C) and bled weekly for one month thereafter. The frequencies of Kb-OVA tetramer⁺, CD44⁺, CD8⁺ cells at different times after immunization are shown. Asterisk indicates statistically significant differences between mice immunized with RAP-treated DC and normally activated DCs (p<0.05). (E) CFSE-labeled DCs were treated as indicated and injected subcutaneously into mice. On indicated days, the total number of CFSE⁺CD11c⁺ DCs within draining or non-draining LNs (NDLN) were calculated and are displayed (n=3-5 per group per day). Asterisks indicate statistically significant differences between RAP-treated and control DC groups (p<0.05). Data are representative of 3 individual experiments.

FIG. 6. Rapamycin enhances the ability of DCs to induce therapeutic anti-tumor immunity. (A) Mice inoculated with tumors on Day 0 were each immunized once subcutaneously with 5×10⁵ DCs treated as indicated on Day 3 and then monitored every three days for tumor growth. Tumor volumes were measured at each time point. The kinetics of tumor growth for each individual mouse in the experiment (n=10 per group) is plotted. 19 days after immunization, mice were sacrificed and tumors were excised, photographed (B) and tumor volume (C) and mass (D) were calculated. Data from all individual mice in the experiment are shown, and mean values illustrated by horizontal bars in (C) and (D). Asterisks show statistically significant differences (p<0.05). (E) Tumor single-cell suspensions were analyzed by FACS and the frequencies of Kb-OVA tetramer⁺CD8⁺ T cells within the CD45⁺ gates are shown. Data are concatenated from all tumors for each mouse group. All tumor experiments were repeated at least three times with similar results.

### DETAILED DESCRIPTION

Antigen presenting cells are recognized within two broad categories generally referred to as professional and non-professional APCs, both types of which can be employed in the present compositions and methods. Professional APCs internalize antigens efficiently by either phagocytosis or receptor-mediated endocytosis and then display a fragment of the antigen, bound to a class II MHC molecule, on their membrane. This type of APC includes dendritic cells (DCs), macrophages, certain B-cells, and certain activated epithelial cells. The “non-professional” APCs do not constitutively express the MHC class II, but do so upon stimulation by certain cytokines, thereby allowing the to function as APCs. Non-professional APCs include fibroblasts, thymic epithelial cells, thyroid epithelial cells, glial cells, pancreatic beta cells, and vascular endothelial cells. An APC as used herein can also be an APC that was induced to differentiate from a stem cell or other progenitor cell.

To obtain antigen presenting cells (APCs) suitable for use in the present methods, one can isolate APCs from blood samples. For example, human DCs can be isolated directly from a sample of human blood. More specifically, for clinical applications and treatment as described further below, blood from a patient can be subjected to leukophoresis to obtain an enriched population of circulating peripheral blood mononuclear cells (PBMCs), which can be further purified as indicated below. APCs can also be obtained from tissue. A third source of APCs is tissue culture. APC subsets can be differentiated in vitro from blood monocytes according to methods known in the art.

In the studies described below, human myeloid DCs were isolated from human blood using MACS positive selection beads. Such purification methods can be used for clinical applications and treatment, as can negative selection or cell sorting. Blood cells were obtained by reverse flushing filters in sterile HBSS. PBMCs were obtained by centrifugation of blood samples over Ficoll-Paque Plus (density 1.077 g/mL) (GE Healthcare). Myeloid DCs were enriched using the BDCA-1 Positive Selection DC Islation Kit (Miltenyi Bioscience) per the manufacturer's instructions. Freshly isolated DC (1×10⁵ cells/well in 200 μl) were cultured in complete RPMI 1640 medium containing 10% FCS, 100 U/mL penicillin/streptomycin, and supplemented with 20 ng/mL GM-CSF. DCs were stimulated simultaneously with 1 μg/mL R848 (TLR agonist) in the presence or absence of mTOR inhibitors (both used at 100 nM). Any one or more of these techniques and compositions (e.g., RPMI 1640 supplemented as described here) can be used in the present methods.

In preparing activated DCs for treating patients, the degree of purity within the DC population can vary from about 80-99 percent (or more). For example, at least or about 75%, 80%, 85%, 90%, or 95% or more of the population of cells exposed to an inhibitor of mTOR, an agonist of a TLR, and a cancer-related antigen can be DCs. Our own studies are consistent with literature reports that mTOR inhibition has a positive effect on the activation of myeloid DCs but a negative effect on the activation of classically defined monocyte-derived human DCs (i.e. monocytes cultured for 7 days in GM-CSF+IL-4 culture conditions). Different subsets of APCs cultured from monocytes can be established by the addition of other cytokines during differentiation (e.g. TNF-alpha, IFN-gamma, IL-6, PGE-2, and others). While the effect of mTOR inhibition on all of the varieties of APCs that can be differentiated from human blood cells has not been thoroughly characterized, it is our belief that any variety of DC differentiated from blood cells (e.g., human blood cells) will be useful in the methods described herein and activated DCs generated from such cells are within the scope of the present invention.

Inhibitors of mTOR: Numerous inhibitors of mTOR (and various combinations thereof) can be used in the present culture methods in order to generate activated DCs. These include rapamycin and its analogs, including sirolimus, everolimus, ridaforolimus, temsirolimus, umirolimus, and zotarolimus) and, broadly, the class of ATP-competitive inhibitors of mTOR function (e.g. Torin1 and Torin2). Other useful inhibitors include EGCG, caffeine, curcumin, and resveratrol. However, these may be less preferable as they may exert non-specific effects. Furthermore, inhibitors of signaling molecules upstream of mTOR its (e.g. inhibitors of Phosphatidylinositide 3-kinases and Protein Kinase B) show similar results to mTOR inhibitors in our experimental system regarding their effect on APC activation.

TLR Agonists: Antigen presenting cell activation can be achieved by targeting any one or more of the Toll-like receptors as described herein in the context of the present methods. Anti-TLR antibodies can also be used (including variants and antigen-binding fragments thereof). TLR ligands suitable for inclusion in the present cultures (and in the methods of DC activation generally) include TLR2, TLR3, TLR4, TLR5, TLR7/8, and TLR9 ligands; any ligand (whether naturally occurring or not) that binds one or more of these TLRs can be used in the present methods for activation of DCs. Other TLR agonists include lipopolysaccharide (LPS), optionally from E. coli. Other agents that do not target a TLR can also be used in addition to or in place of a TLR agonist. For example, the activation methods can be carried out by exposing an APC in cell culture to CD40 ligation.

Cancer-related antigens: The cancer-related antigen can be obtained from a patient (e.g., the patient to be treated). For example, the cultured APCs can be cultured with a tumor lysate or other tumor-derived material. However, we expect that cancer-related antigens used in the present methods will more often be recombinant proteins or peptides. Where the cancer-related antigen is expressed only in a given cancer (as opposed to simply being over-expressed in that cancer), the present methods can be used to produce APCs for prophylactic use (e.g., in, or in the context of, a cancer vaccine). These vaccines can be personalized because a given patient can serve as the source of APCs and/or the antigens used in the treatment.

While the present compositions and methods can, as described above, be used prophylactically, they can also be used to treat a patient known to have or believed to have cancer. Any of the present methods can include a step of identifying a patient in need of treatment, and methods by which a patient's cancer is diagnosed or assessed can be carried out before or concurrently with the methods of APC activation described herein. The current invention is also compatible with use in patients at high risk for cancer because of factors such as genetic mutations, family history, known exposure to oncogenic viruses or carcinogens, and positive test results suggesting cancer, such as mammograms, Pap smears, PSA and other antigen assessments.

The compositions of the present invention (e.g., pharmaceutical compositions) can be optimized to provide an effective or maximum feasible concentration of activated APCs together with an excipient at the site of the cancer. The route of administration can vary depending upon the site of the tumor growth and other factors. Intravenous administration is contemplated as this route of administration can provide the activated APCs systemically (which is useful in the treatment of not only a primary tumor but also secondary metastases). Intravenous and other systemic treatments are also useful in treating cancers of the blood (lymphoma, leukemia and the like). Other routes of administration include but are not limited to subcutaneous, transdermal, intramuscular, intravitreal, epidural, intrathecal, intracerebral, intraperitonial, intravaginal, intracavernous, extra-amniotic, transmucosal, and intratumoral administration. The compositions including activated APCs can be administered by injection, insufflation, infusion or implantation of reservoir devices that release the activated APCs contained within them over time. Administration of the activated APCs may be performed alone or in combination with other appropriately formulated therapeutics.

The composition comprising activated APCs may be in the form of a solution, a suspension, an emulsion, or a lyophilized powder that would be reconstituted prior to use. The compositions may also be configured in unit dosage forms (e.g., in single-dose ampoules), or in vials (or other containers) containing several doses. As noted, the compositions can be contained with a delivery device (e.g., a pre-filled syringe or an implantable devices), and such devices and kits including them are within the scope of the present invention. The activated APCs can also be formulated together with microspheres, microcapsules, nanoparticles, liposomes, and the like.

The contemplated dosage forms and formulations can include a pharmaceutically acceptable excipient or an adjuvant. The excipient can be present in an amount of about 1-99% by weight of the total weight of the composition. Suitable excipients include but are not limited to water, 1,3-butanediol, Ringer's solution, isotonic sodium chloride solution, aqueous dextrose or glycerol solutions, balanced salt solutions, and the like, and they may be adjusted to a suitable pH by addition of an appropriate amount of a suitable acid, base or a buffer. Other suitable excipients may include 1-60% w/w of propylene glycol, buffered saline solution, or the like, and these additives are well known to those skilled in the art (see, e.g., Encyclopedia of Pharmaceutical Technology, eds. J. Swarbrick and J. C. Boylan, 1988-1999, Marcel Dekker, New York; Remington: The Science and Practice of Pharmacy (20th ed.), ed. A. R. Gennaro, Lippincott Williams & Wilkins, 2000). Suitable adjuvants include but are not limited to aluminum gels, aluminum salts and squalene. Other adjuvants, suitable for use in animals, may additionally include but are not limited to Freund's complete adjuvant, Freund's incomplete adjuvant and virosome. The formulations may also contain a preservative such as methyl, ethyl or n-propyl p-hydroxybenzoate. Other included excipients may be solutions, solvents, diluents, suspending agents, solubilizing agents, stabilizing agents, pH-adjusting agents, tonicity adjusting agents, dispersing agents, dissolution enhancing agents, dispersion media, delay agents etc., as known in the art.

### EXAMPLES

### Example 1

### Inhibiting mTOR Promotes Dendritic Cell Activation and Enhances Therapeutic Autologous Vaccination in Mice

Summary: Dendritic cells (DCs) are potent inducers of T cell immunity and autologous DC vaccination holds promise for the treatment of cancers and chronic infectious diseases. In practice, however, therapeutic vaccines of this type have had mixed success. Here, we show that brief exposure to inhibitors of mechanistic Target Of Rapamycin (mTOR) in DCs during the period that they are responding to TLR agonists makes them particularly potent activators of naïve CD8+ T cells, and able to enhance control of B16 melanoma in a therapeutic autologous vaccination model in the mouse. The improved performance of DCs in which mTOR has been inhibited is correlated with an extended lifespan following activation and prolonged, increased expression of costimulatory molecules. Therapeutic autologous vaccination with DCs treated with TLR agonists plus the mTOR inhibitor rapamycin results in improved generation of antigen-specific CD8⁺ T-cells in vivo and improved anti-tumor immunity compared to that observed with DCs treated with TLR agonists alone. These findings define mTOR as a molecular target for augmenting DC survival and activation and document a novel pharmacologic approach for enhancing the efficacy of therapeutic autologous DC vaccination.

Introductory comments: Dendritic cells (DCs) are professional antigen presenting cells responsible for initiating adaptive immune responses (Barton and Medzhitov, Curr. Opin. Immunol., 14:380-383, 2002; Steinman and Hemmi, Curr. Top. Microbiol. Immunol., 311:17-58, 2006). They can be generated from precursor cells in vitro and are of great interest for their potential use in autologous vaccine therapies for cancer and chronic infectious diseases (Palucka et al., Immunol. Rev. 220:129-150, 2007; Palucka et al., Curr. Opin. Immunol. 22:258-263, 2010). Autologous DC vaccines have exhibited limited success clinically (Palucka et al., Immunol. Rev. 220:129-150, 2007; Palucka et al., Curr. Opin. Immunol. 22:258-263, 2010), and the short lifespan of activated DCs is recognized as one obstacle to this promising therapeutic approach (Kim et al., Immunol. Lett. 122:58-67, 2009; Kim et al., Immunol. Lett. 134:47-54, 2010). While genetic approaches to modulating DC lifespan and function can enhance DC vaccine potency in animal tumor models (Kim et al., Immunol. Lett. 122:58-67, 2009; Kang et al., J. Gene Med. 11:889-898, 2009; Okada et al., Gene Ther. 12:129-139, 2005), pharmacological approaches for improving DC immune activity in the context of vaccine therapies are desirable for reasons of clinical feasibility.

One of the central nutrient sensing pathways, controlling a diverse array of cellular responses including cell activation, metabolism, and survival, is governed by mechanistic target of rapamycin (mTOR) (Gulati and Thomas, Biochem. Soc. Trans. 35:236-238, 2007; Kapahi et al., Cell Metab. 11:453-465, 2010; Meijer and Codogno, Nat. Cell Biol. 10:881-883, 2008; Sengupta et al., Mol. Cell. 40:310-322, 2010). mTOR is activated by the PI3K/Akt signaling pathway, which is downstream of a number of growth factor receptors as well as TLRs, and we have previously reported that this signaling axis is critically involved in orchestrating the metabolic demands necessary for DC activation (Krawczyk et al., Blood 115:4742-4749, 2010). Inhibition of mTOR by rapamycin, a macrolide product of the bacterium Streptomyces hygroscopicus, is widely reported to extend the lifespan of eukaryotic cells and organisms (Bjedov et al., Cell Metab 11:35-46, 2010; Bonawitz et al., Cell Metab 5:265-277, 2007; Harrison et al., Nature 460:392-395, 2009). Based on this evidence, we hypothesized that mTOR could play a regulatory role in controlling DC lifespan and activation following TLR stimulation.

We decided to directly examine the role of mTOR in DCs during the activation process following exposure to TLR agonists. We have found that inhibiting mTOR during activation considerably extends the lifespan of DCs, and this is accompanied by enhanced and prolonged costimulatory molecule expression following activation. These phenotypes effectively increase the window of time during which DCs are able to interact with and stimulate antigen-specific T cell responses. Consistent with their increased lifespan and prolonged activation kinetics, DCs activated in the presence of mTOR inhibitors induce enhanced primary antigen-specific CD8⁺ T cell responses and stronger and more effective anti-tumor responses in a therapeutic vaccination treatment model. These findings suggest a novel approach for potentiating the efficacy of autologous DC vaccination for the therapeutic treatment of cancers.

Mice and reagents: C57B1/6 mice were purchased from The Jackson Laboratory, and re-derived stocks were maintained at the Trudeau Institute under specific pathogen-free conditions under protocols approved by the Institutional Animal Care and Use Committee. Lipopoly-saccharide (LPS; Escherichia coli serotype 0111:B4) was from Sigma-Aldrich and used at 100 ng/ml. Pam2CSK4 (1 μg/mL), R848 (1 μg/mL), and CpG (250 ng/mL), and Rapamycin (100 nM) were purchased from Invivogen. KU 0063794 (100 nM) was purchased from Tocris Biochemicals. All antibodies for FACS analysis were from BD Bioscience except for anti-CD11c and CD40, which were purchased from eBioscience. Ovalbumin (250 μg/mL endotoxin-free egg white) was prepared in lab. Kb-OVA tetramers were produced by the Molecular Biology Core Facility at the Trudeau Institute.

Mouse DC culture, retroviral transduction, purification, and activation: Bone marrow-derived DCs were generated as described (Inaba et al., Proc. Natl. Acad. Sci. USA 90:3038-3042, 1993). Briefly, bone marrow cells were differentiated in the presence of GM-CSF (20 ng/mL) in complete DC media (RPMI containing 10% fetal calf serum, 100 U/mL penicillin/streptomycin, and 2 mM L-glutamine) for 6 days. Retroviral transduction of DCs was accomplished as described previously (Sun et al., J. Immunol. 180:1655-1661, 2008). Sequences for Luciferase or mTOR short hairpin RNAs (shRNAs) were obtained from Open Biosystems and cloned into LMP retroviral vectors. Recombinant retroviruses were obtained after the transfection of 293T packaging cells with the use of Lipofectamine (Invitrogen); retrovirus-containing supernatants were collected 48 hours after transfection and used for spin infection (2500 rpm, 2 hours) of day 2 and 3 bone marrow DC cultures in 6-well plates. After 6 days in culture with GM-CSF, DCs were harvested, and transduction efficiency was assessed by human CD8 expression via FACS; typical transduction rates were around 80-90%. On day 6 of culture, DCs were washed in complete DC media and were pulsed as indicated with media alone, rapamycin, LPS, or rapamycin+LPS. Where applicable, cells were pulsed with 250 μg/mL OVA. For retroviral transduction assays, DCs were purified from cultures using CD11c⁺ or human CD8⁺ selection (as indicated) with MACS bead sorting (Miltyeni Biotech) according to the manufacturer's protocol. For IL-10 receptor blocking studies, anti-IL10 receptor monoclonal antibody 1B1.3A and control antibody (HRPN) were purchased from BioXCell.

Human DC Culture: Human myeloid DCs were isolated from human blood using MACS positive selection beads. In brief, filters from hospital blood donors were generously donated by the CVPH Medical Center in Plattsburgh, N.Y. Blood cells were obtained by reverse flushing filters in sterile HBSS. PBMCs were obtained by centrifugation of blood samples over Ficoll-Paque Plus (density 1.077 g/mL) (GE Healthcare). Myeloid DCs were enriched using the BDCA-1 Positive Selection DC Islation Kit (Miltenyi Bioscience) per the manufacturer's instructions. Freshly isolated DC (1×10⁵ cells/well in 200 W) were cultured in complete RPMI 1640 medium containing 10% FCS, 100 U/mL penicillin/streptomycin, and supplemented with 20 ng/mL GM-CSF. DCs were stimulated with 1 μg/mL R848 in the presence or absence of rapamycin or KU (both used at 100 nM). At indicated times, DCs were harvested and analyzed by FACS for maturation markers.

Metabolism Assays: Glucose and Lactate levels in the media after indicated stimulation conditions were measured using the Glucose Assay Kit and Lactate Assay Kit from Eton Bioscience, Inc. per the manufacturer's instructions.

Western blotting: Transduced DCs were MACS-purified based on expression of human CD8. Cell lysate preparation, sodium dodecyl sulfate-polyacrylamide gel electrophoresis, electrophoretic transfer, immunoblotting, and development with enhanced chemiluminescence were accomplished as described previously (Jones et al., Immunity 27:268-280, 2007).

Cytokine measurements and flow cytometry: DC supernatants were analyzed for IL12, TNFα, and IL-10 by FACS using the Cytometric Bead Array Mouse Inflammation Kit (BD Bioscience) per the manufacturer's instructions.

In vitro T-cell responses: For staggered DC activation experiments, DCs were pulsed with indicated treatments for 24 hours and then the media changed to normal growing media (no TLR agonist or mTOR inhibitor). After 24 hour activation, DC was changed daily prior to T cell co-culture to prolong survival of activated DCs. DCs were co-cultured for 4 days at a 1:5 ratio with CFSE-labeled CD8⁺ selected OT-I splenocytes (MACS) on day 2, 3, or 4 following initial DC activation.

Tumor Challenge Experiments: For all tumor studies, mice were challenged with 1×10⁵ Ova-expressing B16 melanoma cells intradermally on the peritoneal surface. For therapeutic vaccine studies, mice were challenged with tumor on Day 0 and then received autologous DC transfer subcutaneously in the left footpad on Day 3 after tumor challenge. Mice were monitored for tumor growth periodically, and tumor sizes were measured with digital calipers (Fisher Scientific). At the time of harvest, mice were sacrificed and tumor was excised with final tumor volumes and weights measured. For analysis of tumor infiltrating cells, tumors were mechanically dissociated in HBSS with a 1 mL syringe stopper and passed through 70 μm cell strainers to obtain single cell suspensions of tumor content.

Statistics: For analysis of CFSE-labeled DCs in the draining LN, 2-way ANOVA was performed on log-transformed data. For analysis of tetramer positive cells in the blood of mice, 2-way ANOVA was performed on data that were transformed by √(percent +0.5) as appropriate for percentage data with very low values (Steel and Torrie, Principles and Procedures of Statistics, with Special Reference to the Biological Sciences, McGraw-Hill, New York, 1960). Tumor volumes were analyzed using Student's t-test on log-transformed values. Tumor weights were first classified by whether or not they exceeded the lower limit of detection for the assay (10 mg) then analyzed using Fisher's exact test.

Declaration of Ethical Compliance with Standards for Study of Human Subjects: Human DCs and monocytes were collected from white blood cells destined to be discarded after being removed by filtration from blood collected for other purposes. The white blood cells were provided to us as samples that were identified by numbers only.

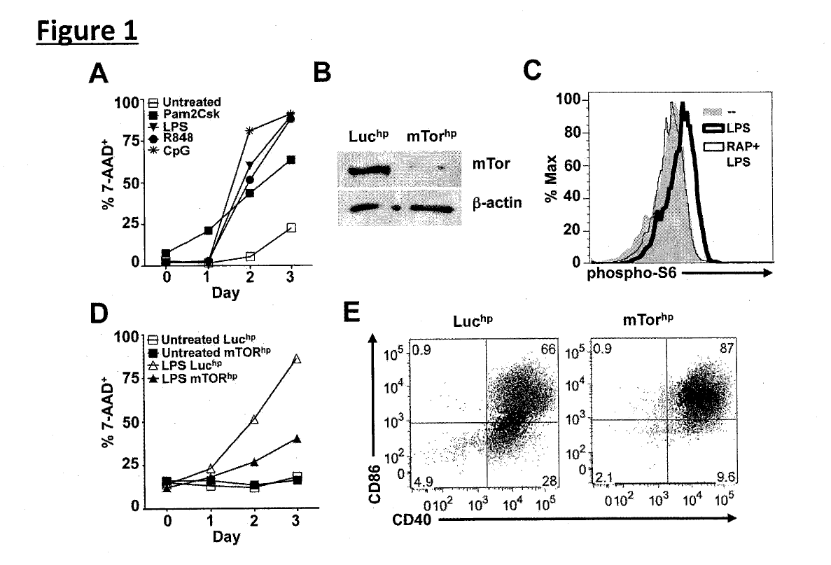

mTOR regulates lifespan and activation of DCs following stimulation by TLR agonists: DC lifespan is dramatically reduced following activation by TLR agonists (FIG. 1A) (Kamath et al., Blood 100:1734-1741, 2002; Matsue et al., J Immunol 162:5287-5298, 1999). We directly assessed the role of mTOR in DC activation and longevity using retroviral transduction of shRNAs targeting mTOR or luciferase (mTOR^{hp} and Luc^{hp} respectively) into bone marrow cells being cultured in GM-CSF (Krawczyk et al., Blood 115:4742-4749, 2010). Transduced DCs emerged equally well from Luc^{hp} (control) and mTOR^{hp} cultures, and mTOR protein expression levels were substantially reduced in DCs transduced with mTOR^{hp} (FIG. 1B). Retroviral transduction alone did not induce a mature phenotype in unstimulated DCs. mTOR signaling, measured as phosphorylation of one of its targets, S6 kinase, is activated by stimulation with LPS and inhibited by rapamycin (FIG. 1C) (Weichhart et al., Immunity 29:565-577, 2008). We therefore asked whether mTOR knockdown would affect the lifespan, or ability to become activated, of DCs responding to the TLR4 agonist LPS. We observed that mTOR deficiency attenuated cell death induced by LPS activation (FIG. 1D), indicating that TLR-driven mTOR signaling is an important determinant of DC lifespan following activation. Furthermore, mTOR knockdown resulted in higher percentages of LPS-stimulated DCs becoming CD40⁺ CD86⁺ (FIG. 1E), demonstrating that TLR-driven mTOR signaling negatively regulates costimulatory molecule expression following LPS activation. This was not a reflection of CD40⁺ CD86⁻ cells being less viable than CD40⁺ CD86⁺ cells, since we were unable to detect differences in 7-AAD staining in these two populations.

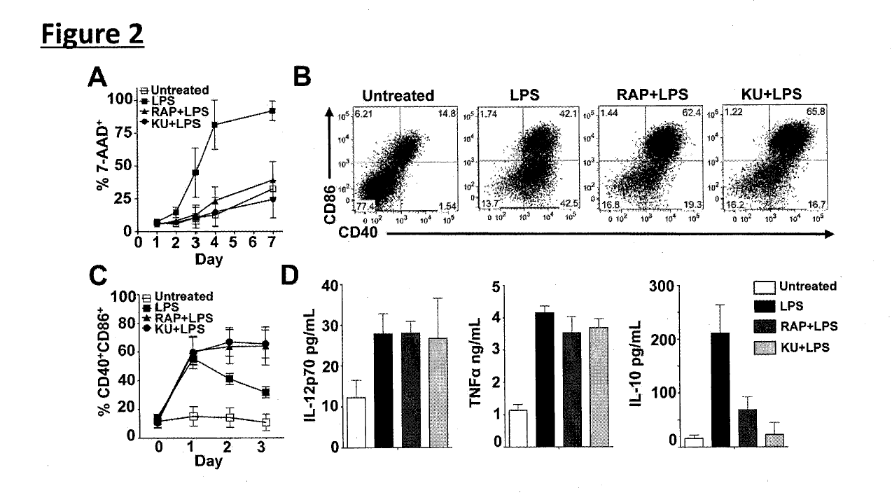

We next tested whether treatment of DCs with rapamycin, a clinically-utilized inhibitor of mTOR signaling (McMahon et al., J Am Soc Nephrol 22:408-415, 2011), or the synthetic ATP-competitive inhibitor of mTOR, KU 0063794 (KU), could recapitulate the phenotypes observed in DCs transduced with mTOR^{hp}. Consistent with the outcome of the mTOR^{hp} experiments, mTOR inhibitors prolonged DC lifespan following activation with LPS (FIG. 2A) or with agonists for TLR2, TLR7/8, and TLR9 (data not shown). Rapamycin and KU treatment alone did not impact the viability of unstimulated DCs (data not shown). Additionally, mTOR inhibition by rapamycin or KU during LPS stimulation resulted in an increase in the percentage of DCs expressing CD40 and CD86 (FIG. 2B), and prolonged the expression of these costimulatory molecules in response to LPS (FIG. 2C). mTOR inhibitors were observed to similarly stabilize the expression of CD80, MHC-I, and MHC-II on LPS-activated DCs.

mTOR functions through two, functionally distinct, signaling complexes: mTORC1, which is sensitive to direct disruption by rapamycin treatment, and mTORC2, which is insensitive to the direct inhibitory effects of rapamycin but can be regulated by mTORC1 activity in some contexts (Sarbassov et al., Curr. Opin. Cell Biol. 17:596-603, 2005; Julien et al., Mol. Cell. Biol. 30:908-921, 2010). mTORC1 signaling regulates protein translation through its interaction with p70S6 kinase and 4E-BP1 while mTORC2 signaling is thought to be involved in regulating cytoskeleton dynamics and negatively regulating Akt signaling via phosphorylation of Ser473 (Sarbassov et al., Curr. Opin. Cell Biol. 17:596-603, 2005; Sarbassov et al., Science 307:1098-1101, 2005; Weichhart and Saemann, Trends Immunol. 30:218-226, 2009). We observed that activation induced Akt Ser473 phosphorylation was inhibited by both rapamycin and KU. Therefore our experiments are unable to distinguish between the role of mTORC1 versus mTORC2 since both of the inhibitors that we utilized reduce mTORC2-mediated Akt phosphorylation.

Consistent with previous reports, mTOR inhibitors did not negatively affect the production of the pro-inflammatory cytokines IL-12p70 and TNFα by LPS-activated DCs, but did inhibit IL-10 production (FIG. 2D) (Haidinger et al., J. Immunol. 185:3919-3931, 2010; Ohtani et al., Blood 112:635-643, 2008). We found that DCs pulsed with ovalbumin in the presence of LPS and rapamycin were highly competent to process and present antigen to CD8⁺ and CD4⁺ T cells in vitro. Thus, mTOR inhibition in DCs during TLR stimulation protects them from activation-associated cell death, and allows them to retain an activated phenotype for prolonged periods without compromising proinflammatory cytokine production or their ability to stimulate T cells in vitro. These effects could not be explained by the observed reduction in IL-10 production, since inhibition of IL-10 signaling using anti-IL-10R antibody did not recapitulate the effects of mTOR inhibition on either lifespan or the duration of costimulatory molecule expression.

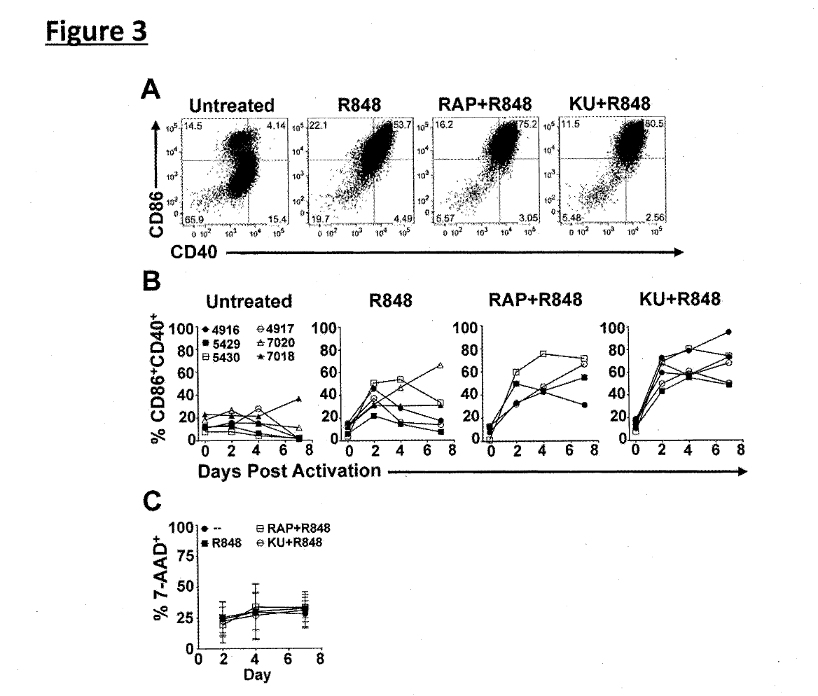

A recent report indicated that, consistent with our findings in mouse BMDCs, rapamycin is able to augment costimulatory molecule expression in human myeloid DCs (Haidinger, et al., J. Immunol. 185:3919-3931, 2010). We examined this directly and found that treatment with rapamycin or KU allowed enhanced costimulatory molecule expression in response to stimulation with the TLR agonist R848 in human myeloid DCs (FIG. 3A). Moreover, enhanced expression of CD40 and CD86 was prolonged as in the mouse DCs when mTOR was inhibited (FIG. 3B). Unlike our results with murine BMDCs, we did not observe the same activation-associated cell death in human myeloid DCs as a result of TLR stimulation (FIG. 3C). However, these findings demonstrate that the enhancing effects of mTOR inhibition on the stability of costimulatory molecule expression observed in mouse DCs is recapitulated in human myeloid DCs, and that this phenotype is not intrinsically dependent on prolonging post-activation survival in these cells.

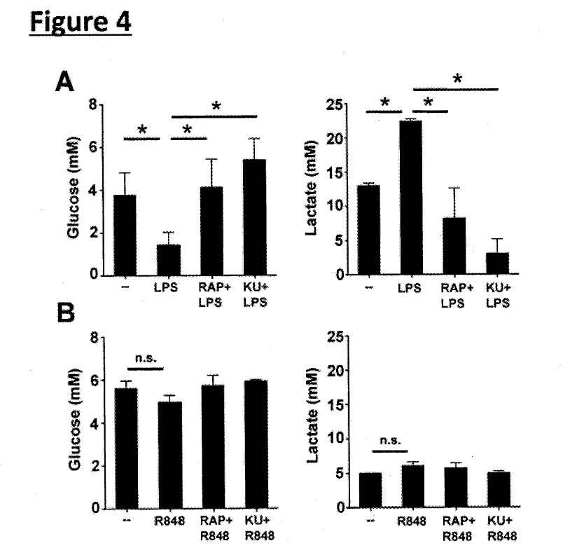

mTOR promotes commitment to glycolytic metabolism following exposure to LPS in mouse but not human DCs: We recently showed that the metabolism of mouse DCs switches away from oxidative phosphorylation towards aerobic glycolysis following activation by TLR agonists (Krawczyk et al., Blood 115:4742-4749, 2010). We found that the death of DCs following stimulation with TLR agonists is in part due to the fact that they are glucose-dependent and able to rapidly exhaust available glucose, and perhaps other nutrients, in tissue culture medium (Krawczyk et al., Blood 115:4742-4749, 2010). Consequently, daily feeding with glucose in vitro is able to extend the lifespan of activated DCs (Krawczyk et al., Blood 115:4742-4749, 2010). Because high levels of glucose consumption generally correlate with shorter lifespan, and mTOR is documented to control the induction of glycolytic metabolism (Duvel et al., Mol. Cell. 39:171-183, 2010; and Csibi and Blenis, BMC Biol. 9:69, 2011), we reasoned that mTOR inhibitors might extend the lifespan of activated DCs by limiting their dependence on glucose. Consistent with this, we found that LPS-induced increases in glucose consumption and the production of lactate (the end product of glycolysis) 48 hours after activation were profoundly diminished by mTOR inhibitors (FIG. 4A). In contrast, neither the use of glucose, nor the production of lactate by human myeloid DCs were affected by exposure to TLR agonists (FIG. 4B), and mTOR inhibitors had no measurable effects on either of these metabolic parameters. These findings may help explain the differences in survival of activated mouse vs. human DCs in our system (FIG. 2A vs. FIG. 3C).

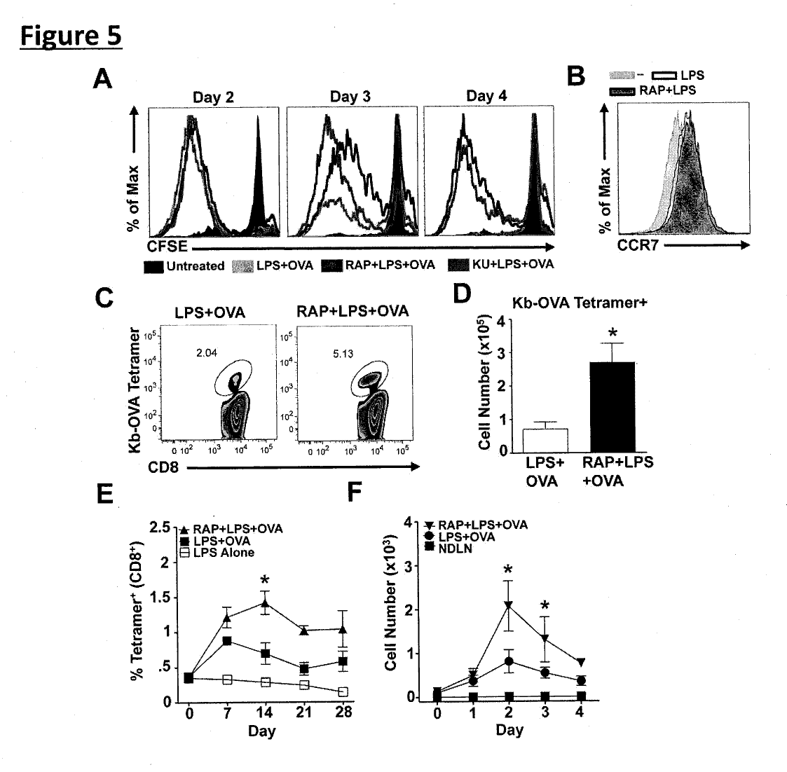

mTOR is a negative regulator of the ability of DCs to activate T cells: The increased duration of costimulatory molecule expression in DCs activated in the presence of mTOR inhibitors indicated that cells treated in this way might be able to continue to activate T cells at times when DCs activated in the absence of mTOR inhibitors are no longer able to do so. To directly test this, we stimulated DCs with LPS and OVA in the presence or absence of mTOR inhibitors, and initiated co-cultures with CD8⁺ OVA-specific OT-I cells on days 2, 3, or 4 after activation; DCs were thoroughly washed to remove drugs, antigen and TLR agonists prior to addition to these co-cultures and equal numbers of DCs were added to each co-culture condition. To focus this analysis on the survival-independent effects of mTOR inhibition, we performed daily media changes of DCs prior to co-culture with T cells, which minimized cell death; this is consistent with previous work from our laboratory demonstrating that media supplementation can extend the lifespan of LPS-activated DCs (Krawczyk et al., Blood 115:4742-4749, 2010). As anticipated, the ability of control DCs activated with LPS and OVA to stimulate T cells deteriorated over time following activation (FIG. 5A). In contrast, DCs activated in the presence of mTOR inhibitors, which were as capable as control DCs of stimulating T cell proliferation at day 2, retained their ability to stimulate T cells even at day 4 after DC activation (FIG. 5A). Thus, the ability of DCs to remain activated and capable of stimulating T cells following exposure to antigen and TLR-agonists is markedly enhanced by the inhibition of mTOR.

DCs in which mTOR is inhibited have an enhanced capacity to induce therapeutic CD8 T cell responses in vivo: The ability of mTOR inhibition to prolong DC activation and T-cell stimulatory capacity following maturation in vitro suggested that inhibition of DC mTOR signaling might enhance the ability of these cells to induce T cell responses in vivo. To test this possibility, we stimulated DCs with LPS, LPS plus OVA, or rapamycin plus LPS plus OVA for 6 hours, then washed them extensively and injected them subcutaneously into mice. Endogenous OVA-specific CD8⁺ T-cell responses in draining LNs were monitored by tetramer staining 7 days following DC transfer. Rapamycin-treated DCs were not impaired in their ability to migrate to LNs draining sites of injection (data not shown and discussed below), consistent with the fact that they increased expression of CCR7 in response to LPS equivalently to DCs that were stimulated with LPS in the absence of rapamycin (FIG. 5B). The addition of rapamycin to DCs during the time that they were pulsed with LPS plus OVA enabled them to induce stronger immune responses as measured by LN expansion, and the frequency (FIG. 5C) and number (FIG. 5D) of Kb-OVA tetramer positive CD8⁺ T cells in reactive LNs. We also detected antigen-specific CD8⁺ T cells in blood and spleen of immunized mice, and these cells were present in higher frequencies in the mice that received rapamycin-treated DCs. We observed that frequencies of circulating antigen-specific CD8⁺ T cells increased between days 7 and 14 post-immunization with rapamycin-treated DCs, whereas contraction of the antigen-specific population occurred during this period in mice immunized with DCs that had not been treated with rapamycin (FIG. 5E). Additionally, we observed higher numbers of injected rapamycin-treated DCs than control DCs in reactive LNs draining sites of immunization on days 3 and 4 after DC transfer (FIG. 5F), suggesting that the in vitro survival advantage conferred by mTOR inhibition (FIG. 2A) may also be at play in vivo. Based on in vitro data (FIG. 5A), we would expect the rapamycin-treated DCs persisting at days 3 and 4 to be able to continue to activate T cells in the in vivo setting.

Our principal focus was on the ability of mTOR inhibition to augment DC activity during the primary T cell response following autologous DC transfer. However, we were also interested in testing whether vaccination with DCs activated in the presence of mTOR inhibitors could induce the development of a population of antigen-specific memory CD8+ T cells. Immunization with DCs pulsed with LPS plus OVA or with rapamycin plus LPS plus OVA resulted in the establishment of robust memory CD8⁺ T cell populations that, 4-5 weeks after priming, were highly capable of responding to challenge infection with OVA-expressing Listeria monocytogenes, or of mediating protection against challenge with OVA-expressing B16 melanoma cells. Taken together, our data demonstrate that DCs activated in the presence of rapamycin are capable of inducing enhanced CD8 T cell primary expansion and contraction during the primary phase of the response, and the establishment of a population of memory CD8+ T cells that can be recalled by re-exposure to antigen.

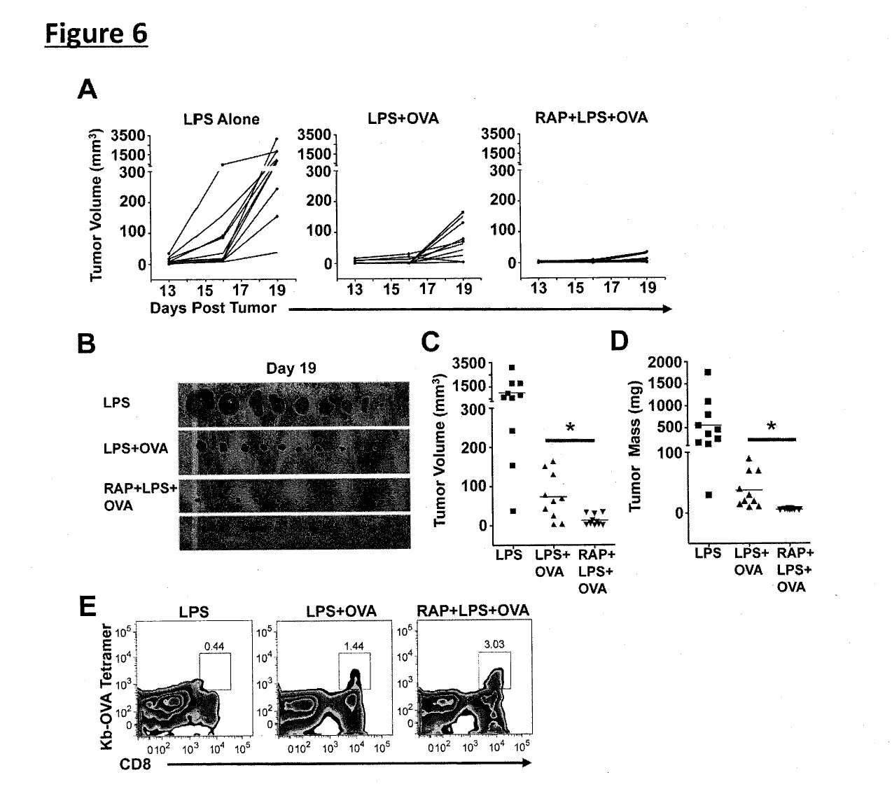

Therapeutic autologous DC vaccination has significant promise for cancer treatment (Higano et al., Cancer 115:3670-3679, 2009). We reasoned that the advantages conferred to DCs by mTOR inhibition might enhance their ability to induce therapeutic anti-tumor immunity. To test this hypothesis, we intradermally inoculated mice with OVA-expressing B 16 melanoma cells. Three days later, we vaccinated tumor recipients with DCs that had been pulsed in vitro for 6 hours with LPS, with LPS plus OVA, or with rapamycin plus LPS plus OVA. Mice that received the rapamycin-treated DCs following tumor inoculation had increased frequencies of OVA-specific CD8⁺ T cells in the blood 1 week after DC vaccination compared to mice that received DCs that had not been treated with rapamycin (data not shown), indicating that the in vivo benefit conferred by rapamycin-treated DCs is not sensitive to systemic tumor-mediated immune suppression. Consistent with a protective role for CD8⁺ T cells in this system, the kinetics of tumor growth were substantially delayed in mice vaccinated with DCs stimulated with LPS plus OVA (FIG. 6A). Most importantly, there was a highly significant reduction in tumor burden, obvious macroscopically (FIG. 6B), and as measured by tumor volume (FIG. 6C) and mass (FIG. 6D) at the time of sacrifice in mice that received the rapamycin-treated DCs. As anticipated, regardless of whether they were treated with rapamycin or not, DCs pulsed with LPS plus OVA conferred significant therapeutic advantage over the control treatment of DCs that had been pulsed with LPS without antigen. The improved therapeutic anti-tumor effect of immunization with rapamycin-treated DCs was associated with a doubling in the frequency of antigen-specific CD8⁺ tumor-infiltrating lymphocytes observed in single cell suspensions of harvested tumors (FIG. 6E). These data provide evidence that mTOR-inhibition in DCs during in vitro stimulation with antigen and TLR-agonists prior to transfer into a recipient can significantly improve the subsequent in vivo therapeutic potential of these cells

Discussion: Here we describe mTOR inhibition during DC activation as a novel strategy to improve autologous DC vaccination in a murine melanoma tumor model. We demonstrate that pharmacological inhibition of mTOR prolongs the lifespan of TLR-activated mouse DCs and extends the time period over which they exhibit an activated phenotype. The combined effect of these two processes results in a dramatic difference in outcome. By 2-4 days following activation in the absence of mTOR inhibitors, most DCs are dead, and those that remain alive are no longer expressing high levels of costimulatory molecules. In contrast, by 2-4 days following activation in the presence of mTOR inhibitors, most of the DCs within the starting population are alive and continuing to express high levels of costimulatory molecules. Not surprisingly in light of these findings, DCs activated in the presence of mTOR inhibitors induce larger CD8 T cell responses both in the local lymphoid compartments and systemically when adoptively transferred into naïve host animals. This enhanced CD8 T cell response induced by DCs in which mTOR is inhibited correlates with a more pronounced protective effect against an aggressive melanoma in a therapeutic vaccination model. Strikingly, these beneficial effects of mTOR inhibition on DCs are induced by a brief 6-hour exposure to mTOR inhibitors at the time of the addition of TLR agonists to these cells. The fact that short-term in vitro pharmacological intervention can have beneficial effects on the kinetics human DC activation status suggests that it may be technically feasible to translate this approach for enhancing autologous DC vaccination into a clinical setting.

We observed identical effects on DC lifespan, costimulatory molecule expression, and cytokine production with either rapamycin- or KU-treatment. However, because both rapamycin and KU treatment showed comparable inhibitory effects on the phosphorylation of Akt Ser473, a downstream target of mTORC2 activity, we cannot distinguish from these data which mTOR signaling complex is the predominate effector complex regulating DC activation and lifespan in our system. Future studies genetically targeting the role of mTORC1 vs. mTORC2 in DC activation and survival will be an interesting avenue for further investigation.

Elucidating the role of mTOR during DC activation in response to TLR agonists is complicated by its established role as central mediator of signaling initiated by growth factor receptors, including that for GM-CSF, an important DC differentiation and survival factor (Sarbassov et al., Curr Opin Cell Biol 17:596-603, 2005; Woltman et al., Blood 101:1439-1445, 2003). Work in human monocyte-derived DCs has demonstrated that mTOR inhibition can disrupt GM-CSF signaling in these cells, inhibiting differentiation and leading to apoptosis (Woltman et al., Blood 101:1439-1445, 2003). Consistent with this, the inclusion of pharmacological inhibitors of mTOR in mouse bone marrow DC cultures did negatively affect the final yield of DCs (data not shown). In contrast, the suppression of mTOR expression by retrovirally introduced mTOR-targeting shRNA did not prevent GM-CSF-driven differentiation of mouse DCs from bone marrow, possibly because the hairpin is not expressed until after the DC precursors have finished proliferating in response to GM-CSF (Krawczyk et al., J. Immunol. 180:7931-7937, 2008). Taken together, these data indicate that mTOR signaling may be important for different functions at different times over the lifespan of a DC. The fact that mTOR inhibition actually promoted increased DC survival following activation by TLR agonists emphasizes the emerging concept that the effects of mTOR regulation of DC metabolism and growth factor signaling may be highly context specific. Understanding the nuances of the functional role of mTOR in DCs has potential to provide important insights about the underlying cell biology controlling activation and survival signals in these cells.

One of the challenges raised by the findings reported here showing adjuvant-like effects of inhibiting mTOR in DCs is to reconcile our data with the expansive literature on the use of mTOR inhibitors to promote immune tolerance Inhibition of mTOR by rapamycin is reported to support the induction of tolerogenic DCs (Taper et al., Am. J. Transplant 5:228-236, 2005; Turnquist et al., J. Immunol. 181:62-72, 2008) and to inhibit both Flt3 ligand and GM-CSF-driven DC differentiation in vitro (Haidinger et al., J. Immunol. 185:3919-3931, 2010; Woltman et al., Blood 101:1439-1445, 2003; Hackstein et al., Blood 101:4457-4463, 2003; Sathaliyawala et al., Immunity 33:597-606, 2010). In contrast, our data strongly support an emerging view that, in certain contexts, mTOR inhibitors administered simultaneously with TLR stimulation can enhance the activation of DCs (Haidinger et al., J Immunol 185:3919-3931, 2010; Ohtani et al., Blood 112:635-643, 2008). While other studies have indicate that mTOR inhibitors act differently in different subsets of APCs, we are the first to show a beneficial effect of this class of drugs on antigen presentation to T lymphocytes and to apply it to an in vivo model of a clinical application.

The underlying explanation for this apparent disagreement may lie in the details of how experiments are performed and in the nature of the DCs being tested. Based on the role of mTOR in signaling by a number of growth factors and cytokines (Sengupta et al., Mol. Cell. 40:310-322, 2010; Sarbassov et al., Curr. Opin. Cell Biol. 17:596-603, 2005; Woltman et al., Blood 101:1439-1445, 2003), we speculate that specific culture conditions during in vitro generation of DCs may determine the effect of mTOR inhibition on these cells. For example, Flt3 ligand-differentiated DCs may respond differently to mTOR inhibitors during TLR activation than GM-CSF-differentiated DCs. We speculate that by restricting mTOR inhibition specifically to the initial phases of TLR activation, we can modulate DC activity without inducing the deleterious effects that may be associated with disrupted growth factor signaling in some DC subtypes.

We previously proposed that the prolonged increase in aerobic glycolysis in activated DCs serves to rapidly generate ATP, while conserving the metabolic intermediates such as amino acids and fatty acids that we predicted would be important for the complete enactment of the activation process (Krawczyk et al., Blood 115:4742-4749, 2010). Nevertheless, we observed that mTOR-inhibition in the context of TLR activation potently inhibits metabolic commitment to glycolysis, but does not affect TLR-mediated activation, insofar as antigen processing and presentation, IL-12 and TNFα production, costimulatory molecule expression, cellular migration, and DC:T cell interactions are concerned. Thus it is clear that TLR-agonist mediated DC activation does not require commitment to aerobic glycolysis when mTOR is inhibited. Indeed, our findings indicate that sustained glycolysis subsequent to TLR activation is associated with rapid cell death and that restricting glucose consumption by mTOR inhibition prolongs the lifespan of these cells without compromising their ability to stimulate T cells in vitro or in vivo. A strong association between glucose consumption and cell death is widely reported in the literature and restricting glucose usage and caloric intake leads to increased cellular and organismal longevity (Kapahi et al., Cell Metab. 11:453-465, 2010; Bjedov et al., Cell Metab. 11:35-46, 2010; Bonawitz et al., Cell Metab. 5:265-277, 2007; Lee et al., Cell Metab. 10:379-391, 2009; Li et al., FASEB J. 24:1442-1453, 2010; Li and Tollefsbol, PLoS One 6:e17421, 2011). Interestingly, the strong metabolic shift to aerobic glycolysis upon LPS stimulation was not observed in human myeloid DCs, which may be an important factor in the different survival kinetics of activated human and mouse DCs in our systems. However, mTOR inhibition augmented and prolonged costimulatory molecule expression in both human and mouse DCs, indicating that mTOR may be regulating DC survival and costimulatory molecule expression by two different mechanisms. The underlying mechanism by which inhibition of mTOR allows DCs to enact their activation program in the absence of a switch to glycolysis as well as the apparent differences in metabolic regulation of DCs between human and mouse are current focuses of research in our laboratory.

One of the intriguing findings of our study is the disparity in sensitivity of IL-10 versus IL-12p70/TNFα production to mTOR inhibition. The mTOR substrate 4EBP1 drives cap-dependent translation (Carayol et al., J Biol Chem 283:8601-8610, 2008; Choo et al., Proc Natl Acad Sci USA 105:17414-17419 2008; Kumar et al., EMBO J 19:1087-1097, 2000; Mamane et al., Oncogene 25:6416-6422, 2006), so despite the fact that similar observations have been reported previously regarding the specific inhibition of IL-10 production in DCs by mTOR ablation Haidinger et al., J Immunol 185:3919-3931, 2010; Ohtani et al. Blood 112:635-643, 2008), it was surprising to us that whilst TLR-agonist induced IL-10 production was inhibited by rapamycin, the production of IL-12p70 and TNFα remained unaffected. This finding raises the possibility that translation of IL-12p70 and TNFα transcripts can be initiated by cap-independent processes (Shatsky et al., Mol. Cell. 30:285-293, 2010). In light of our observations on the effects of mTOR on the surface expression of costimulatory molecules, we speculate that normal cellular dynamics like vesicular trafficking may also have slowed in rapamycin-treated cells. This is consistent with the fact that mTOR controls the expression of genes involved in cholesterol and fatty acid synthesis (Csibi and Blenis, BMC Biol. 9:69, 2011).

There is an ongoing interest in biomedical research in harnessing the immune-stimulatory properties of DCs for immune intervention against tumors. The Food and Drug Administration recently approved Provenge, the first autologous cellular immunotherapy for use in cancer patients (Drake, Cancer J. 17:294-299, 2011), which marks an important hallmark in advancing the use of cellular immunotherapy strategies in the clinic. Furthermore, DC vaccination strategies have shown significant promise in early-phase clinical trials in human melanoma patients (Ridolfi et al., Melanoma Res., 2011). Our studies using a mouse model of melanoma outline a potential new strategy for augmenting DC activation in the context of therapeutic autologous vaccination. Because brief exposure to mTOR inhibitors in vitro enhances the activation phenotype in both mouse and human DCs, we believe that our findings may be of significant relevance to ongoing clinical research aimed at improving the potency of DC therapeutic vaccines.

Our data define mTOR signaling as a new molecular target for augmenting DC survival and activation. We show that mTOR inhibition extends the lifespan of DCs, and enhances the expression of key costimulatory molecules involved in the initiation of adaptive immune responses. Ongoing studies are aimed at further understanding the consequences of mTOR inhibition in the context of the beneficial effects on DCs reported here. The fact that mTOR inhibitors, including rapamycin, are approved for use in human patients, and that our approach requires that DCs be exposed to rapamycin only in vitro prior to transfer into recipients, increases the feasibility of potentially translating this simple strategy to clinical settings. In summary, our studies demonstrate a novel pharmacological approach for temporally extending DC lifespan, prolonging DC activation, and improving the outcome of autologous DC vaccination for experimentally induced cancer in mice.

What is claimed is:

1. A method of making an activated antigen presenting cell (APC), the method comprising exposing an antigen presenting cell, in cell culture, to: an inhibitor of the mTOR (mammalian target of rapamycin) signaling axis; an agonist that mediates activation of the APC; and a cancer-related antigen.

2. The method of claim 1, wherein the APC is mammalian or mammalian in origin.

3. The method of claim 2, wherein the APC is human or human in origin.

4. The method of claim 1, wherein the inhibitor of the mTOR signaling axis inhibits mTOR or another protein in the molecular complexes mTORC1 or mTORC2.

5. The method of claim 4, wherein the inhibitor of the mTOR signaling axis inhibits mTOR.

6. The method of claim 5, wherein the inhibitor of mTOR is rapamycin or a rapamycin analog.

7. The method of claim 6, wherein the rapamycin analog is sirolimus, everolimus, ridaforolimus, temsirolimus, umirolimus, or zotarolimus.

8. The method of claim 1, wherein the inhibitor of the mTOR signaling axis targets a molecule upstream of mTOR signaling.

9. The method of claim 8, wherein the inhibitor of the mTOR signaling axis inhibits a phosphatidylinositide 3-kinase or Protein Kinase B.

10. The method of claim 1, wherein the agonist that mediates activation of the APC is an antibody moiety that specifically binds a Toll-like receptor (TLR).

11. The method of claim 1, wherein the agonist that mediates activation of the APC is a TLR ligand.

12. The method of claim 11, wherein the TLR ligand is lipopolysaccharide (LPS), optionally obtained or derived from E. coli.

13. The method of claim 1, wherein the agonist that mediates activation of the APC is an agonistic CD40 antibody or Type I Interferon stimulation.

14. The method of claim 1, wherein the cancer-related antigen is an indicator of an epithelial cancer.

15. The method of claim 1, wherein the cancer related antigen is: A3, AFP, AKAP-4, ALK, androgen receptor, B7H3, Bcr-abl, BORIS, BR-1, BRAC1, BRAC2, carbonic anhydrase IX, CEA, cyclin B1, CYP1B1, EGFRviii, EpCAM, EphA2, ERG, ESO-1, ETV6-AML, FAP, fos-related antigen 1, fucosyl GMT, GD2, GD3, GMe ganglioside, GloboH, gp100, HER-2/neu, HMWMAA, HPV E6 or E7, hTERT, LCK, legumain, LMP2, MAC-CT-1, MAD-CT-2, MAGE, MAGE A1, MelanA/MART1, mesothelin, ML-IAP, MUC1, MYCN, NA17, NY-RGS5, NY-Ras-mutant, OY-TEST, p53, Page4, PAP, PAX3, PAX5, PDGFR-beta, PLAC1, polysialic acid, proteinase3, PSA, PSCA, PSMA, RhoC, SART3, sLe(a), sperm protein 17, a sperm fibrous sheath protein, SSX2, STn, survivin, Tie 2, Tn, TRP-1, TRP-2, tyrosinase, VGFR2, WT1, or XAGE1.

16. The method of claim 1, wherein the APC is exposed to the inhibitor of the mTOR signaling axis and/or to the agonist that mediates activation of the APC either prior to the time or concurrent with the time the APC is exposed to the cancer-related antigen.

17. An activated antigen presenting cell (APC) made by the method of claim 1.

18. A pharmaceutically acceptable composition comprising the activated APC of claim 17.

19. A kit comprising the activated APC of claim 17 and instructions for use.

20. A method of treating a patient who has cancer, the method comprising administering to the patient an activated APC, wherein the APC is made by the method of claim 1.
